SEQUENCE LISTING (10 sequences)

SEQ ID NO: 1 - protein, 19 aa, Artificial Sequence
  SEQ ID NO 1  is a peptide chemically synthesized by the laboratory.
  SITE (19)..(19): The c-terminal amidation of SEQ ID NO 1.
KKKKKKKKWRKWLKWLAKK

SEQ ID NO: 2 - protein, 19 aa, Artificial Sequence
  SEQ ID NO 2  is a peptide chemically synthesized by the laboratory.
  MISC_FEATURE (15)..(15): Xaa is  beta-naphthylalanine (Nal)
  SITE (19)..(19): The c-terminal amidation of SEQ ID NO 2.
KKKKKKKKWRKWLKXLAKK

SEQ ID NO: 3 - protein, 16 aa, Artificial Sequence
  SEQ ID NO 3  is a peptide chemically synthesized by the laboratory.
  MISC_FEATURE (13)..(13): Xaa is  beta-naphthylalanine (Nal)
  SITE (16)..(16): The c-terminal amidation of SEQ ID NO 3.
KKKKKKWRKWLKXLAK

SEQ ID NO: 4 - protein, 19 aa, Artificial Sequence
  SEQ ID NO 4  is a peptide chemically synthesized by the laboratory.
  SITE (19)..(19): The c-terminal amidation of SEQ ID NO 4.
KKKKRRKKWLKWLKWLKKK

SEQ ID NO: 5 - protein, 19 aa, Artificial Sequence
  SEQ ID NO 5  is a peptide chemically synthesized by the laboratory.
  MISC_FEATURE (15)..(15): Xaa is  beta-naphthylalanine (Nal)
  SITE (19)..(19): The c-terminal amidation of SEQ ID NO 5.
KKKKRRKKWLKWLKXLKKK

SEQ ID NO: 6 - protein, 16 aa, Artificial Sequence
  SEQ ID NO 6  is a peptide chemically synthesized by the laboratory.
  MISC_FEATURE (13)..(13): Xaa is  beta-naphthylalanine (Nal)
  SITE (16)..(16): The c-terminal amidation of SEQ ID NO 6.
KKKKRRWLKWLKXLKK

SEQ ID NO: 7 - protein, 19 aa, Artificial Sequence
  SEQ ID NO 7  is a peptide chemically synthesized by the laboratory.
  SITE (19)..(19): The c-terminal amidation of SEQ ID NO 7.
KKKKRRKKWRKWLKWLAKK

SEQ ID NO: 8 - protein, 19 aa, Artificial Sequence
  SEQ ID NO 8  is a peptide chemically synthesized by the laboratory.
  MISC_FEATURE (15)..(15): Xaa is  beta-naphthylalanine (Nal)
  SITE (19)..(19): The c-terminal amidation  of SEQ ID NO 8.
KKKKRRKKWRKWLKXLAKK

SEQ ID NO: 9 - protein, 16 aa, Artificial Sequence
  SEQ ID NO 9  is a peptide chemically synthesized by the laboratory.
  MISC_FEATURE (13)..(13): Xaa is  beta-naphthylalanine (Nal)
  SITE (16)..(16): The c-terminal amidation of SEQ ID NO 9.
KKKKRRWRKWLKXLAK

SEQ ID NO: 10 - protein, 10 aa, Artificial Sequence
  SEQ ID NO 10  is a peptide chemically synthesized by the laboratory.
  MISC_FEATURE (7)..(7): Xaa is  beta-naphthylalanine (Nal)
  SITE (10)..(10): The c-terminal amidation of SEQ ID NO 10.
KKKKRRXLAK

(19) United States
(12) Patent Application Publication
(10) Pub. No.: US 2025/0250298 A1
(43) Pub. Date: Aug. 7, 2025

(54) ONCOLYTIC PEPTIDE AND USE THEREOF

(71) Applicant: Rise Biopharmaceuticals Inc., Haidian District, Beijing (CN)

(72) Inventors: Jya-Wei Cheng, East District, Tainan (TW); Chih-Lung Wu, East District, Tainan (TW)

(21) Appl. No.: 18/856,501

(22) PCT Filed: Apr. 12, 2022
(86) PCT No.: PCT/CN2022/086220

§ 371 Date: Oct. 11, 2024

(51) Int. Cl. C07K 7/08; A61K 38/00; A61P 35/00; C07K 7/06

(52) U.S. Cl. CPC C07K 7/08; A61P 35/00; C07K 7/06; A61K 38/00

(57) ABSTRACT

Compositions for oncolytic peptide therapeutics of cancer are provided. Methods of using such compositions containing the same are also described. Additionally, the oncolytic peptide can be used to inhibit tumor growth and decrease tumor volume.

### CROSS-REFERENCE TO RELATED APPLICATIONS

This application is a national phase entry under 35 U.S.C. § 371 of International Patent Application PCT/CN2022/086220, filed Apr. 12, 2022, designating the United States of America and published as International Patent Publication WO 2023/197129 A1 on Oct. 19, 2023.

### TECHNICAL FIELD

This disclosure relates to an oncolytic peptide for inhibiting tumor growth and resulting in an anti-tumor response.

### BACKGROUND

Cancer imposes a global health burden as it represents one of the leading causes of morbidity and mortality while also giving rise to significant economic burden due to the associated expenditures for its monitoring and treatment.

Cancer cells are characterized by uncontrolled proliferation and the ability to invade surrounding normal tissue or distant sites through homological and/or lymphatic spread. The difficulty for effective treatment of cancer lies in distinguishing between malignant and normal cells of the body. Both are derived from the same source and are very similar, and for this reason, there is no effective recognition by the immune system of the threat of cancer cells.

Despite the advances made in cancer therapy over the past few decades, such as surgery, radiotherapy, chemotherapy and immunotherapy, these therapeutic modalities are still associated with significant side effects. There are still challenges in treating cancers that are chemotherapy or immunotherapy resistant. These represent significant unmet needs.

It is therefore attempted to fill such unmet needs by designing and developing peptide-based cancer therapeutics that are potent, safe and low in production costs. This disclosure addresses several inherent weaknesses in current cancer therapies that are in need of attention.

### BRIEF SUMMARY

Cancer is the most popular disease cause of death in developed countries. If a cancer is diagnosed at an early stage, it is more likely to be treated successfully. Although there has been considerable progress in the diagnosis and treatment of cancer, these treatments are either causing serious side effects or ineffective.

In order to solve the above-mentioned problems, the disclosure provides a new way to treat cancer.

Another aspect of the disclosure provides an effective method to inhibit or reduce cancer cells by administering therapeutically effective amount of the peptides to a patient.

Detailed description of the disclosure is given in the following embodiments with reference to the accompanying drawings.

### BRIEF DESCRIPTION OF THE DRAWINGS

The drawings described herein are for illustration purposes only and are not intended to limit the scope of the present invention in any way.

FIGS. 1A-1B are schematic diagrams of B16-F10 melanoma experiments. The right flank of the mouse was administrated with SEQ ID NO:9, and the left flank of the mouse was untreated (tumor only) in FIG. 1A. FIG. 1B shows that B16-F10 tumor cells were harvested and washed in medium and injected intradermally (ID.) into both flanks of C57BL/6 mice. (3×10⁶B16-F10 cells per mouse/50 μl RPMI-1640). Palpable tumors (20-30 mm³) were injected intrathecally (IT.) with single doses of SEQ ID NO:9 dissolved in PBS (1.0 mg peptide/50 μl PBS) once a day for 5 consecutive days, and the vehicle control was saline only (0.9% NaCl).

FIGS. 2A-2D show that SEQ ID NO:9 significantly inhibited tumor growth in vivo compared with the saline control group. The inhibitory effect was a systemic response, and the distal tumor (left side) was also inhibited (FIGS. 2A-2C). In addition, SEQ ID NO:9 extended the lifespan of tumor-bearing mice (FIG. 2D). FIGS. 2A-2D demonstrate the anti-tumor effects of SEQ ID NO:9 in B16-F10 tumor-bearing mice model. FIG. 2A: Representative images of B16-F10 tumors-bearing mice on day 8 (before treatment) and on day 12 after SEQ ID NO:9 treatment or untreated groups. FIGS. 2B-2C: The changes of tumor sizes on left flanks and on right flanks of B16-F10 tumor-bearing mice. FIG. 2D: The survival curve of SEQ ID NO:9-treated mice compared to untreated controls.

FIGS. 3A-3B show that SEQ ID NO:9 treatment maintained body weight and food intake compared to the control group. FIG. 3A: The body weight of control (wild-type), untreated (B16-F10 tumor only), and SEQ ID NO:9 treated group. FIG. 3B: The percentage of food intake of the control (wild-type), the untreated (B16-F10 tumor only), and the SEQ ID NO:9 treated group.

FIGS. 4A-4B are schematic diagrams of MN-11 fibrosarcoma experiments. FIG. 4A: The right flank of the mouse was administrated with SEQ ID NO:9, and the left flank of the mouse was untreated (tumor only). FIG. 4B shows that MN-11 tumor cells were harvested, and washed in medium and injected subcutaneously (S.C.) into both flanks of C57BL/6 mice (5×10⁵MN-11 cells per mouse/50 μl RPMI-1640). Palpable tumors (100 mm³) were injected (IT.) with single doses of SEQ ID NO:9 dissolved in PBS (1.0 mg peptide/50 μl PBS) once a day for 5 consecutive days, and the vehicle control was saline only (0.9% NaCl).

FIGS. 5A-5D show that the antitumor effects of SEQ ID NO:9 in MN-11 tumor-bearing mice model. FIG. 5A: Representative images of MN-11 tumors-bearing mice before treatment, after SEQ ID NO:9 treatment and untreated controls. FIGS. 5B-5C: The changes of tumor sizes on left flanks and on right flanks of MN-11 tumor-bearing mice. FIG. 5D: The survival curve of SEQ ID NO:9-treated mice compared to untreated controls.

FIGS. 6A-6B show that the SEQ ID NO:9 treatment maintained the body weight and the food intake compared to the control group. FIG. 6A: The body weights of the control (wild-type), the untreated (tumor only), and the SEQ ID NO:9 treated group. FIG. 6B: The percentage of food intake of the control (wild-type), the untreated (tumor only), and the SEQ ID NO:9 treated group.

FIGS. 7A-7B are schematic diagrams of E0771 breast cancer experiments. FIG. 7A: The right flank of the mouse was administrated with SEQ ID NO:9. FIG. 7B shows that E0771 tumor cells were harvested, and washed in medium and injected subcutaneously (S.C.) into the right flanks of C57BL/6 mice. (2×10⁵E0771 cells per mouse/50 μl RPMI-1640). Palpable tumors (75 mm³) were injected (IT.) with single doses of SEQ ID NO:9 dissolved in PBS (1.0 mg peptide/50 μl PBS) once a day for 5 consecutive days, and the vehicle control was saline only (0.9% NaCl).

FIGS. 8A-8C show that the antitumor effects of SEQ ID NO:9 in E0771 tumor-bearing mice model. FIG. 8A: Representative images of E0771 tumors-bearing mice before and after SEQ ID NO:9 treatment, and untreated controls. FIG. 8B: The changes of tumor sizes on right flanks of E0771 tumor-bearing mice. FIG. 8C: The survival curve of SEQ ID NO:9-treated mice compared to untreated controls.

FIGS. 9A-9B show that SEQ ID NO:9 treatment maintained the body weight and the food intake compared to the control group. FIG. 9A: The body weights of the control (wild-type), the untreated (E0771 tumor only), and the SEQ ID NO:9 treated group. FIG. 9B: The percentage of food intake of the control (wild-type), the untreated (E0771 tumor only), and the SEQ ID NO:9 treated group.

### DETAILED DESCRIPTION

While preferred embodiments of the disclosure are shown and described herein, such embodiments are provided by way of example only and are not intended to otherwise limit the scope of the disclosure. Various alternatives to the described embodiments of the disclosure may be employed in practicing the invention.

Unless otherwise defined, all technical terms used herein have the same meaning as commonly understood by one with ordinary skills in the arts of this field to which this disclosure belongs.

The terms “amino acid sequence,” “protein,” “polypeptide” and “peptide” are used interchangeably herein to refer to two or more amino acids, or “residues,” covalently linked by an amide bond or equivalent. Amino acid sequences can be linked by non-natural or non-amide chemical bonds.

In one embodiment, the disclosure provides an oncolytic peptide of formula (I): Xm-(AC1X)n-(DLC2)Xq, wherein X is selected from the group consisting of basic amino acids; wherein A is selected from the group consisting of aromatic amino acids; wherein C1 or C2 is selected from the group consisting of basic amino acids and non-polar amino acids; wherein D is selected from the group consisting of aromatic amino acids and non-natural amino acids; wherein L is leucine; and m is 4 to 8, q is 0 to 2, n is 0 to 2 of formula (I).

In another embodiment, the basic amino acid is selected from the group consisting of lysine, arginine, and histidine.

In another embodiment, the aromatic amino acid is selected from the group consisting of tryptophan, phenylalanine, and tyrosine.

In another embodiment, the non-polar amino acid is selected from the group consisting of leucine, alanine, valine, isoleucine, proline, phenylalanine, methionine, and tryptophan.

In another embodiment, the non-natural amino acid is selected from the group consisting of β-naphthylalanine (Nal), (benzothien-3-yl) alanine (Bal), diphenylalanine (Dip), (4,4′-biphen-yl) alanine (Bip), (anthracen-9-yl) alanine (Ath) and (2,5,7-tri-tert-butyl-indol-3-yl) alanine (Tht).

In another embodiment, a C-terminus of the oncolytic peptide is formed in part by the group of modifications including amidation, acetylation, formylation, hydroxylation, lipid modification, methylation and phosphorylation.

This disclosure further provides a pharmaceutical composition for treating cancer or inhibiting tumor growth, including a therapeutically effective amount of an oncolytic peptide of the disclosure and a pharmaceutically acceptable excipient.

In one embodiment, cancer types include melanoma, fibrosarcoma, prostate cancer, breast cancer, uterine cancer, leukemia, ovarian cancer, endometrial cancer, cervical cancer, colorectal cancer, testicular cancer, lymphoma, rhabdomyosarcoma, neuroblastoma, pancreatic cancer, lung cancer, brain tumor, skin cancer, stomach cancer, oral cancer, liver cancer, laryngeal cancer, gallbladder cancer, thyroid cancer, liver cancer, kidney cancer, or nasopharyngeal carcinoma.

The term “therapeutically effective amount” as used herein means an amount of an oncolytic peptide effective in producing the desired therapeutic response in a particular patient (subject) suffering from cancer. Particularly, the term “therapeutically effective amount” includes the amount of the therapeutic agents, which when administered will achieve the desired therapeutic effects. In the context of the disclosure, the desired therapeutic effects include partial or total inhibition; delay or prevention of the progression of cancer including cancer metastasis; inhibition, delay or prevention of the recurrence of cancer including cancer metastasis; and/or the prevention of the onset or development of cancer in a subject. In respect of the therapeutic amount of the therapeutic agents, i.e., the oncolytic peptides, consideration is also given that the amount of each of the therapeutic agent used for the treatment of a subject is low enough to avoid undesired or severe side effects, within the scope of sound medical judgment. The therapeutically effective amount when used in combination will vary with the age and physical condition of the end user, the severity of cancer, the duration of the treatment, the nature of any other concurrent therapy, the specific type of therapeutic agent employed for the treatment, the particular pharmaceutically acceptable carrier utilized in the pharmaceutical compositions containing the therapeutic agents and other relevant factors.

“Subjects” as used herein are generally human subjects including, but not limited to, cancer patients. The subjects may be male or female and may be of any race or ethnicity. The subjects may be of any age, including newborn, neonate, infant, child, adolescent, adult, and geriatric. Subjects may also include animal subjects, particularly mammalian subjects such as canines, felines, bovines, caprines, equines, ovines, porcines, rodents (e.g., mouse, rat, guinea pig, and hamster), lagomorphs, primates (including non-human primates), etc.

The dose of the oncolytic peptide of the disclosure is appropriately determined depending upon the purpose for therapy, and conditions such as sex, age, weight of the subject, administration route, and severity of the disease.

The term “administration” or “administering” includes routes of introducing the oncolytic peptides of the disclosure to a subject to perform their intended function. The oncolytic peptide of the disclosure can be administered orally, buccally, parenterally, by inhalation spray, rectally, intradermally, transdermally, or topically in dosage unit formulations containing conventional nontoxic pharmaceutically acceptable carriers, adjuvants, and vehicles as desired.

“Treating” or “treatment” as used herein refers to the treating or treatment of a disease or medical condition (such as cancer, tumor, neoplasm conditions) in a subject/patient, such as a mammal (particularly a human) which includes ameliorating the disease or medical condition, i.e., eliminating or causing regression of the disease or medical condition in a subject/patient; suppressing the disease or medical condition, i.e., slowing or arresting the development of the disease or medical condition in a subject/patient; or alleviating the symptoms of the disease or medical condition in a subject/patient.

The term “pharmaceutically acceptable” as used herein means that the carrier, diluent, excipient, and/or salt used in the composition should be compatible with the other ingredients of the formulation, and not deleterious to the recipient thereof. “Pharmaceutically acceptable” also means that the compositions or dosage forms are within the scope of sound medical judgment, suitable for use for a subject such as an animal or human without excessive toxicity, irritation, allergic response, or other problems or complication, and commensurate with a reasonable benefit/risk ratio.

The oncolytic peptide of the disclosure can be administered in a single dose, in multiple doses throughout a 24-hour period, or by continuous infusion. When administered by continuous infusion, the compounds can be supplied by methods well known in the field, such as, but not limited to, intravenous gravity drip, intravenous infusion pump, implantable infusion pump, or any topical routes. Length of treatment will vary depending on many factors. Treatment of the subject with the oncolytic peptide of the disclosure alone or in combination with other agents may continue for the life of the subject.

Additional specific applications of the disclosure include, but are not limited to the following:

### EXAMPLES

The following examples are described to illustrate the application of the disclosure. These examples are provided for the purpose of illustration only and should in no way be considered as being the only examples. Application of the disclosure should include any and all variations that become evident as a result of the descriptions provided herein.

The data of the following EXAMPLES were analyzed by GraphPad Prism. All results of the following EXAMPLES were presented as a mean±SD of at least two independent experiments. The results were analyzed using the one-way ANOVA and Student's t-test. P-values<0.05 were considered statistically significant.

### Example 1

### Oncolytic Peptides

Optionally, in an exemplary embodiment of the disclosure, the oncolytic peptides of the disclosure includes, but are not limited to, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Sequences of SEQ ID NO:1 to SEQ ID NO:10, which can be employed in accordance with the disclosure, are shown in Table 1 as follows.

TABLE 1
Oncolytic peptides sequence

| Name | Sequence | Molecular weight (Da) |
|---|---|---|
| SEQ ID NO: 1 | KKKK-KK-KK-WRK-WLK-WLA-KK-NH₂ | 2567.32 |
| SEQ ID NO: 2 | KKKK-KK-KK-WRK-WLK-Nal-LA-KK-NH₂ | 2578.35 |
| SEQ ID NO: 3 | KKKK-KK-WRK-WLK-Nal-LAK-NH₂ | 2193.83 |
| SEQ ID NO: 4 | KKKK-RR-KK-WLK-WLK-WLK-KK-NH₂ | 2637.41 |
| SEQ ID NO: 5 | KKKK-RR-KK-WLK-WLK-Nal-LK-KK-NH₂ | 2684.4 |
| SEQ ID NO: 6 | KKKK-RR-WLK-WLK-Nal-LKK-NH₂ | 2263.92 |
| SEQ ID NO: 7 | KKKK-RR-KK-WRK-WLK-WLA-KK-NH₂ | 2623.3 |
| SEQ ID NO: 8 | KKKK-RR-KK-WRK-WLK-Nal-LA-KK-NH₂ | 2634.37 |
| SEQ ID NO: 9 | KKKK-RR-WRK-WLK-Nal-LA-K-NH₂ | 2249.85 |
| SEQ ID NO: 10 | KKKK-RR-Nal-LAK-NH₂ | 1351.73 |

### Example 2

### Determination of Half Inhibitory Concentration Via Cytotoxicity of Oncolytic Peptides Against Cancer Cell Lines

The MTT assay is a colorimetric method for measuring the activity of enzymes in living cells that reduces Thiazolyl blue tetrazolium bromide (MTT) to formazan dyes showing a purple color. In this embodiment, a colorimetric 3-(4.5-dimethylthiazol-2-yl)-2.5-diphenyltetrazodium bromide (MTT) viability assay was used to assess the in vitro cytotoxicity of oncolytic peptides.

The half inhibition concentration (IC50) is the concentration required by the oncolytic peptides to reach fifty percent inhibition. In one embodiment, the oncolytic peptides were used for IC50 test via MTT colorimetric assay.

Human lung cancer lines PC9 and A549 and murine pancreatic cancer cell line Panc02 were cultured in RPMI medium supplemented with 10% fetal bovine serum and antibiotic in this embodiment. Additionally, murine hepatoma cell line Hepa 1-6, murine melanoma cell line B16-F10, murine fibrosarcoma cell line MN-11, murine mammary cancer cell line E0771 and human diploid fibroblast (HFW) were cultured in DMEM medium supplemented with 10% fetal bovine serum and antibiotic. Cells were cultured in a humidified incubator containing 5% CO₂ at 37° C.

All cancer cell lines were seeded in a 96-well plate with a concentration of 5000-8000 cells/100 μl/well and incubated for 24 hrs. After medium was removed, 100 μl fresh medium containing peptide (ranging from 50 μM to 0.8 μM) was added to the wells. Following 24 hours incubation, fresh medium with MTT (0.5 mg/ml) was replaced and incubated for 3 hours. After medium/MTT was removed, DMSO was added at 100 μl for dissolving the formazan crystal. Cell survival rate was calculated by measuring the absorbance at 540 nm using a Multi-labeled Microplate Reader (Sunrise™, TECAN).

The data showed that the oncolytic peptides have potent anticancer activities against different cancer cell lines (Table 2).

TABLE 2
Half inhibitory concentrations of the oncolytic peptides tested with cancerous cell lines.

| | IC₅₀^{a}(μM) | | | | | | | | | |
|---|---|---|---|---|---|---|---|---|---|---|
| | SEQ ID | SEQ ID | SEQ ID | SEQ ID | SEQ ID | SEQ ID | SEQ ID | SEQ ID | SEQ 10 | SEQ ID |
| Cell lines | NO: 1 | NO: 2 | NO: 3 | NO: 4 | NO: 5 | NO: 6 | NO: 7 | NO: 8 | NO: 9 | NO: 10 |
| HFW (Human fibroblast) | 16.28 | 10.98 | 9.15 | 20.47 | 11.15 | 7.20 | 12.02 | 7.29 | 3.38 | 488.23 |
| HUVEC (Human Umbilical | 9.35 | 9.03 | 7.61 | 4.66 | 7.23 | 6.84 | 3.95 | 5.6 | 6.32 | N/A |
| Vein Endothelial (Cell) | | | | | | | | | | |
| EO771 (Murine mammary | 16.72 | 10.07 | 6.32 | 13.86 | 12.66 | 10.00 | 6.16 | 3.3 | 2.01 | 55.36 |
| adenocarcinoma) | | | | | | | | | | |
| Hepa I-6 (Murine | | | | | | | | | | |
| hepatocellular carcinoma) | 13.33 | 8.50 | 7.16 | 10.49 | 7.34 | 8.46 | 4.92 | 3.59 | 1.69 | 41.82 |
| B16-F10 (Murine skin | 19.23 | 13.73 | 15.44 | 16.28 | 12.75 | 10.45 | 15.29 | 9.73 | 7.16 | 136.23 |
| malignant melanoma) | | | | | | | | | | |
| MN-11 (Murine | 14.59 | 13.39 | 16.08 | 13.20 | 10.06 | 5.56 | 5.73 | 3.49 | 2.90 | 93.46 |
| fibrosarcoma) | | | | | | | | | | |
| Panc02 (Human pancreatic | 13.36 | 7.17 | 8.43 | 16.01 | 6.62 | 8.81 | 10.63 | 2.663 | 2.66 | 75.23 |
| ductal adenocarcinoma) | | | | | | | | | | |
| PC9 (Human non-small cell | 8.48 | 4.36 | 6.85 | 7.87 | 3.68 | 3.36 | 2.93 | 1.58 | 3.02 | N/A |
| lung cancer cell) | | | | | | | | | | |
| A549 (Human non-small cell | 7.19 | 6.34 | 5.05 | 8.89 | 10.75 | 7.07 | 3.30 | 2.3 | 2.79 | 46.32 |
| lung cancer cell) | | | | | | | | | | |
| C9 (Human oral cancer cell) | 21.04 | 16.00 | 13.20 | 16.56 | 14.24 | 12.65 | 9.06 | 5.30 | 6.43 | 56.83 |
| OECM-1 (Human oral | 18.76 | 15.46 | 15.47 | 18.03 | 16.09 | 16.45 | 10.20 | 5.97 | 6.36 | 35.10 |
| cancer cell) | | | | | | | | | | |
| SAS (Human tongue | 14.73 | 15.82 | 17.05 | 18.58 | 12.13 | 15.56 | 7.67 | 3.98 | 6.03 | 98.43 |
| carcinoma) | | | | | | | | | | |
| HepG2 Cancer Cells | 13.42 | 10.97 | 15.74 | 14.64 | 20.01 | 14.23 | 12.12 | 8.18 | 9.81 | 84.77 |
| (Human hepatocellular | | | | | | | | | | |
| carcinoma) | | | | | | | | | | |
| NCI-N87 (Human gastric | 17.07 | 8.58 | 10.66 | 15.12 | 8.86 | 9.41 | 6.16 | 3.16 | 2.35 | 82.87 |
| carcinoma) | | | | | | | | | | |
| HCT-15 (Human colorectal | 12.21 | 6.60 | 8.89 | 10.77 | 7.59 | 8.03 | 7.13 | 3.16 | 2.42 | 57.62 |
| cancer) | | | | | | | | | | |
| A375 (human malignant | 10.58 | 5.59 | 8.27 | 9.98 | 11.17 | 10.83 | 4.01 | 3.59 | 3.84 | N/A |
| melanoma) | | | | | | | | | | |
| RBC (Human blood cell) | 52.58 | 45.03 | 38.37 | 58.59 | 44.30 | 35.37 | 50.54 | 37.64 | 25.66 | N/A |

^{a}The Cytotoxic activity of peptides given as the mean inhibitory concentration killing 50% of the cells (IC₅₀) measured for cell lines. . .

The anticancer ability ranked in the order SEQ TD NO:7-9>SEQ ID NO:1-3 SEQ ID NO:4-6>SEQ ID NO:10 as disclosed in Table 2. In comparison with the data of half inhibitory concentration, it was discovered that the combination of “KKKKRR,” “WRKWLKWLA,” plus “beta-naphthylalanine (Nal)” in the oncolytic peptide would have better anticancer activity. Further, the length of the peptide sequence connected behind “KKKKRR” is a key factor for anticancer activity.

### Example 3

### Peptide Stability in Serum

Since this kind of peptide is highly toxic, the hope is that the peptide can be decomposed after inducing cancer cells to release danger-associated molecular patterns (DAMPs), and will not stay in the body for too long. After exposure to 25% (vol/vol) bovine calf serum, the residues of peptides were analyzed by liquid chromatography, and the stability at different time points was calculated. The results showed that the peptide of SEQ ID NO:7 decreased by nearly 40% at the fourth hour, and by nearly 56% after eight hours, indicating that the peptide will be decomposed after it interacts with tumor cells and will not accumulate in the human body (Table 3).

TABLE 3
Serum stability results for SEQ ID NO. 7, SEQ ID NO. 8, and SEQ ID NO. 9.

| Time (hr) | 0 | 0.5 | 1 | 2 | 4 | 6 | 8 |
|---|---|---|---|---|---|---|---|
| SEQ ID NO: 7 | 100.00% | 100.80% | 123.30% | 72.10% | 59.50% | 54.40% | 44.40% |
| SEQ ID NO: 8 | 100.00% | 104.90% | 77.40% | 106.20% | 70.40% | 71.30% | 66.50% |
| SEQ ID NO: 9 | 100.00% | 90.70% | 76.20% | 77.90% | 70.90% | 74.20% | 55.80% |

The stability of the oncolytic peptides in serum ranked in the order SEQ ID NO:9>SEQ ID NO:8>SEQ ID NO:7 at 4 hr. The numbers of amino acid residues in the oncolytic peptides had no positive correlation with serum stability. However, serum stability had positive correlation with beta-naphthylalanine (Nal) in the peptide sequence. Further, the stability of the oncolytic peptides in serum ranked in the order SEQ ID NO:9>SEQ ID NO:8>SEQ ID NO:7 at 6 hr. However, the stability of the peptides in serum ranked in the order SEQ ID NO:8>SEQ ID NO:9>SEQ ID NO:7 at 8 hr.

### Example 4

### Animal Experiments

Female C57BL/6 mice (4- to 6-week old) as allogeneic model animals were purchased from BioLASCO Taiwan Co., Ltd. and maintained in a specific pathogen-free and controlled environment. During the experiments, female mice, weighing 18.0-24.0 g each, were kept in groups of 4 to 6 animals per cage under climate-controlled conditions, with 12 h light/dark cycles and an ambient temperature. The mice were housed in an enriched individually ventilated cage (IVC) system with free access to standard rodent chow and water ad libitum. The animals were anesthetized during the experimental procedures with 2.5% isoflurane gas. The animals were monitored daily. All procedures were conducted according to the regulations of Laboratory Animal Care and Use Committee or Group Setup and Management and the law of Animal Protection.

Palpable tumors (for example, 20-30 mm³ for B16-F10 tumor-bearing mice, 100 mm³ for MN-11 tumor-bearing mice, 75 mm³ for E0771 tumor-bearing mice) were injected (IT.) with single doses of SEQ ID NO:9 dissolved in PBS (1.0 mg peptide/50 μl PBS) once a day for 5 consecutive days, and the vehicle control was saline only (0.9% NaCl).

Tumor size was measured 3 times a week using a caliper, and the volume of tumors was calculated using the following formula: Tumor size (mm³)=0.5×(width²×length). The results were shown as volume±SEM. The survival rates of tumor-bearing mice were observed and recorded continuously until all animals died.

### Example 5

### Inhibiting Melanoma Growth in Allograft Animal Model Prolongs Survival In Vivo

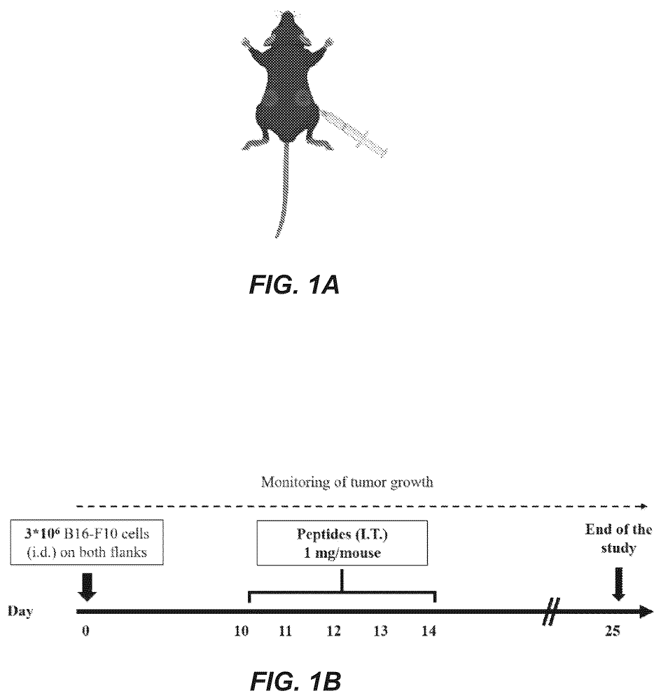

To evaluate the effect of SEQ ID NO:9 in vivo, the B16-F10 allogeneic model was used in C57BL/6 mice (FIGS. 1A-1B). B16-F10 tumor cells were harvested, and washed in medium and injected intradermally (ID.) into both flanks of the C57BL/6 mice. (3×10⁶B16-F10 cells per mouse/50 μl RPMI-1640).

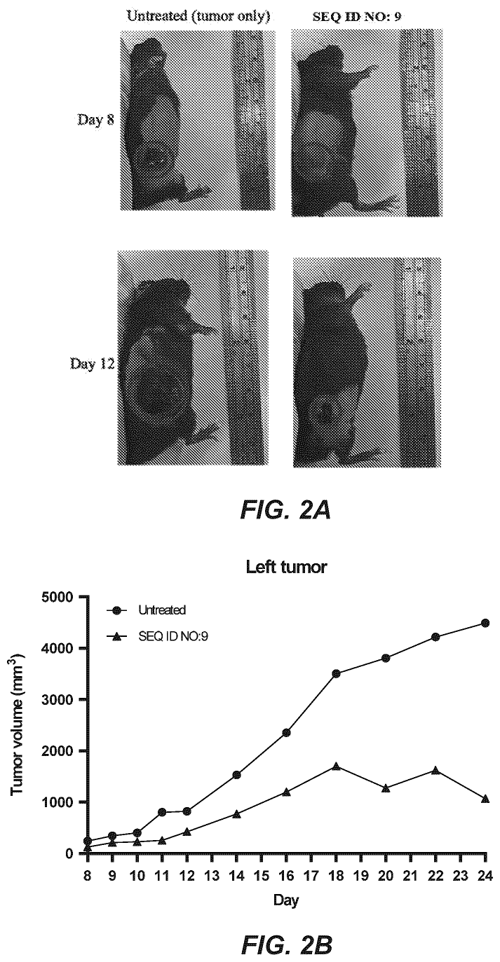

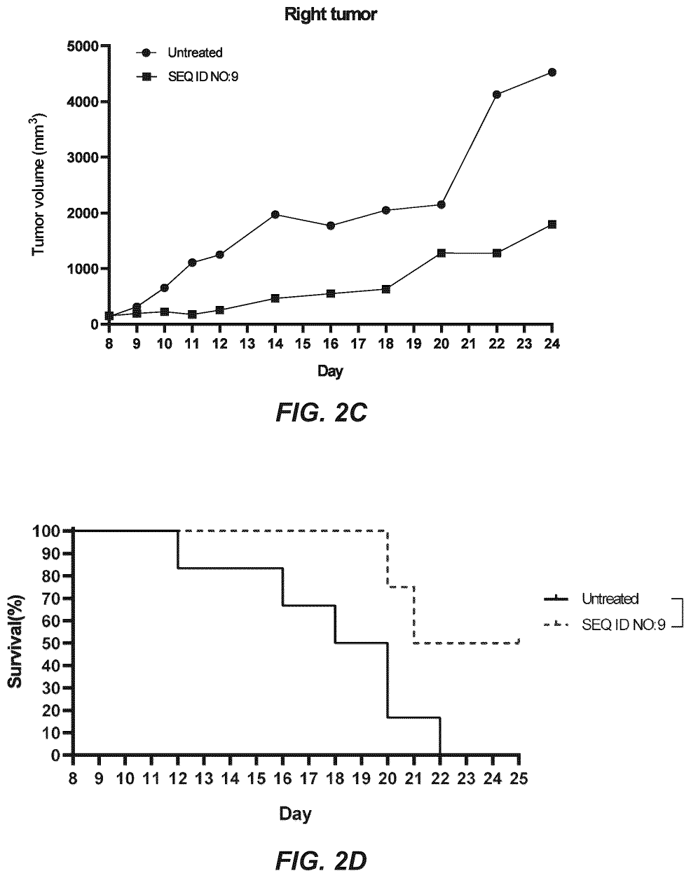

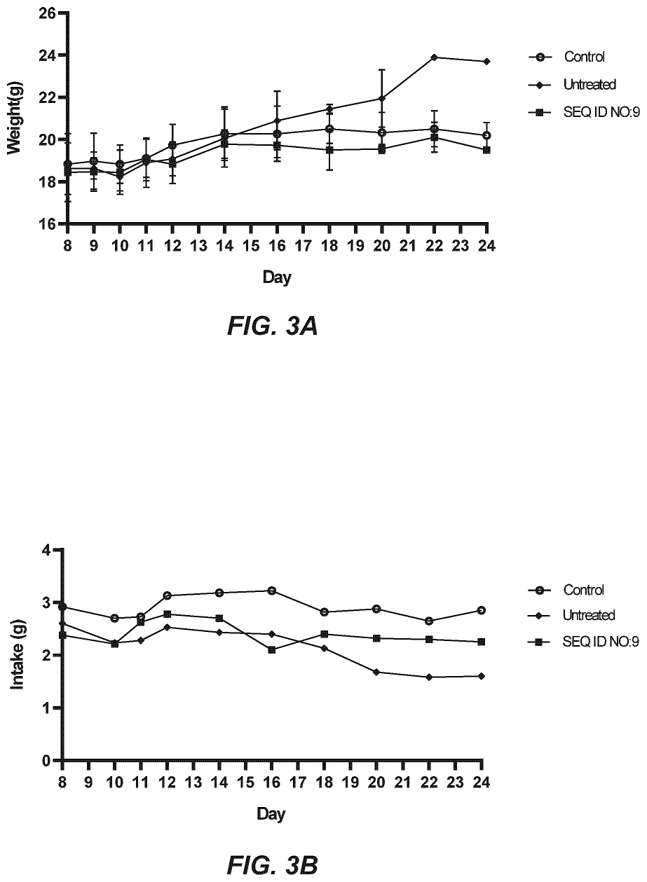

The experiment demonstrated that SEQ ID NO:9 significantly inhibited tumor growth in vivo compared with the saline control group, and the inhibitory effect was a systemic response, and the distal tumor (left side) was also inhibited (FIGS. 2A-2D). Furthermore, treatment with SEQ ID NO:9 did not result in significant changes in body weight or food intake in the mice, implying that SEQ ID NO:9 may not have toxic effects in vivo (FIGS. 3A-3B). The reason for the weight loss after treatment was presumed to be caused by a change in the excessive swelling of the tumor.

### Example 6

### Inhibiting Fibrosarcoma Growth in Allograft Animal Model Prolongs the Survival In Vivo

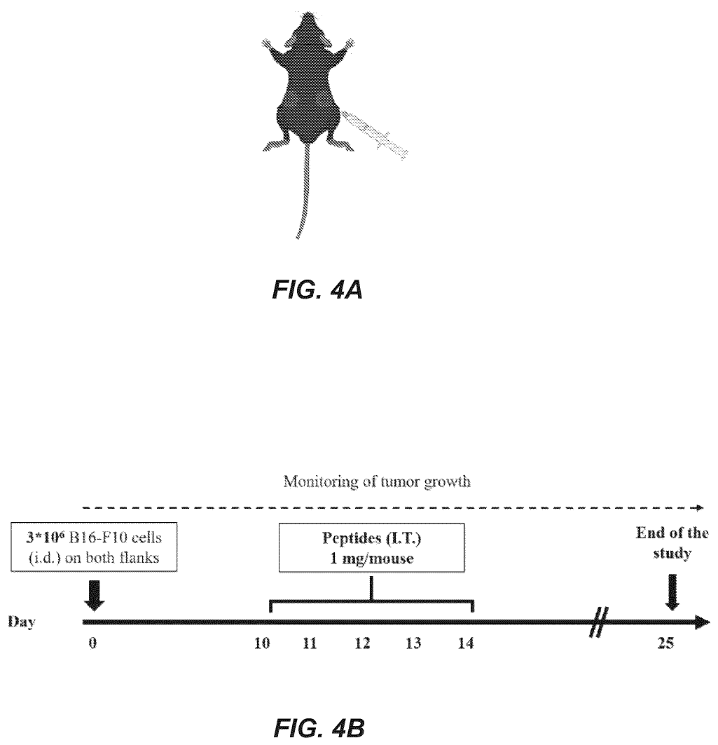

To evaluate the effect of SEQ ID NO:9 in vivo, the embodiment was used the MN-11 allogeneic model in C57BL/6 mice (FIGS. 4A-4B). MN-11 tumor cells were harvested, and washed in medium and injected subcutaneously (S.C.) into the both flanks of C57BL/6 mice. (5×10⁵ MN-11 cells per mouse/50 μl RPMI-1640).

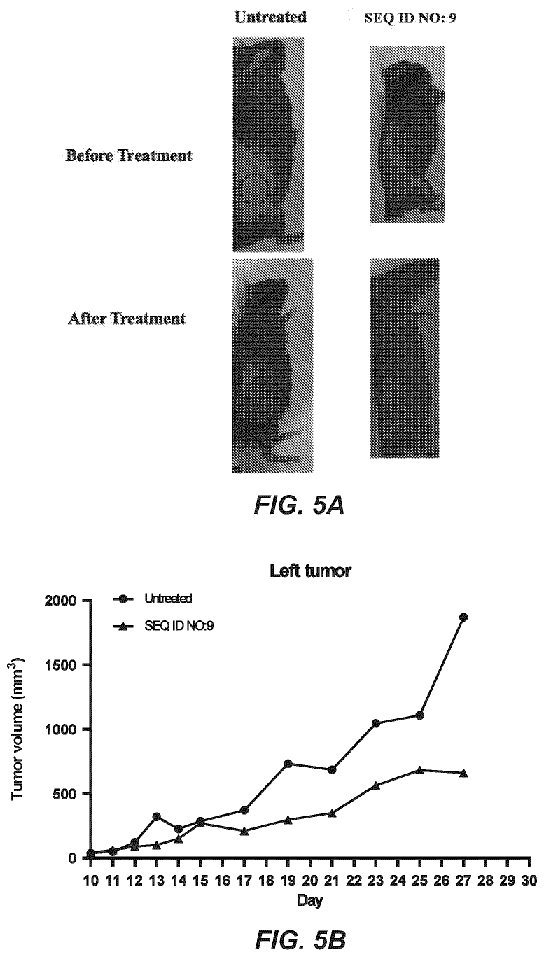

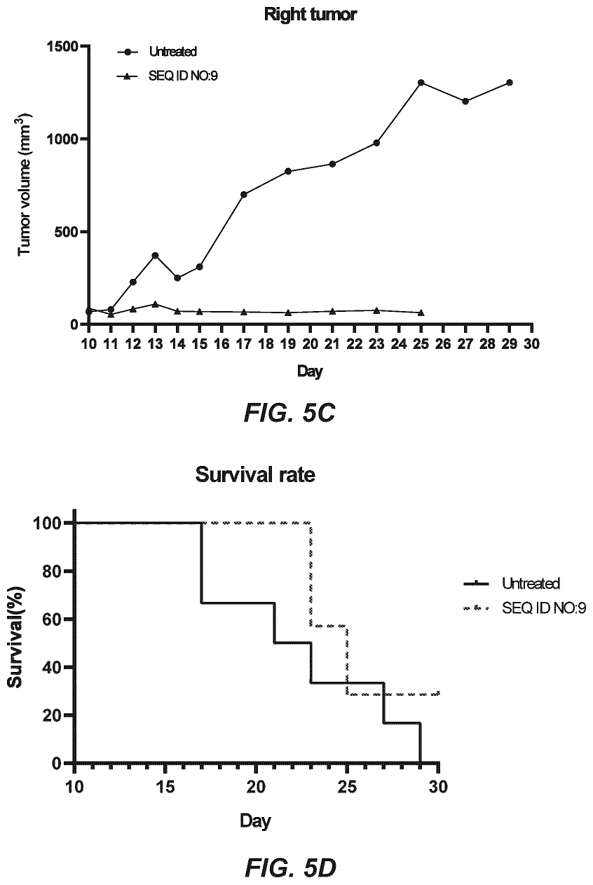

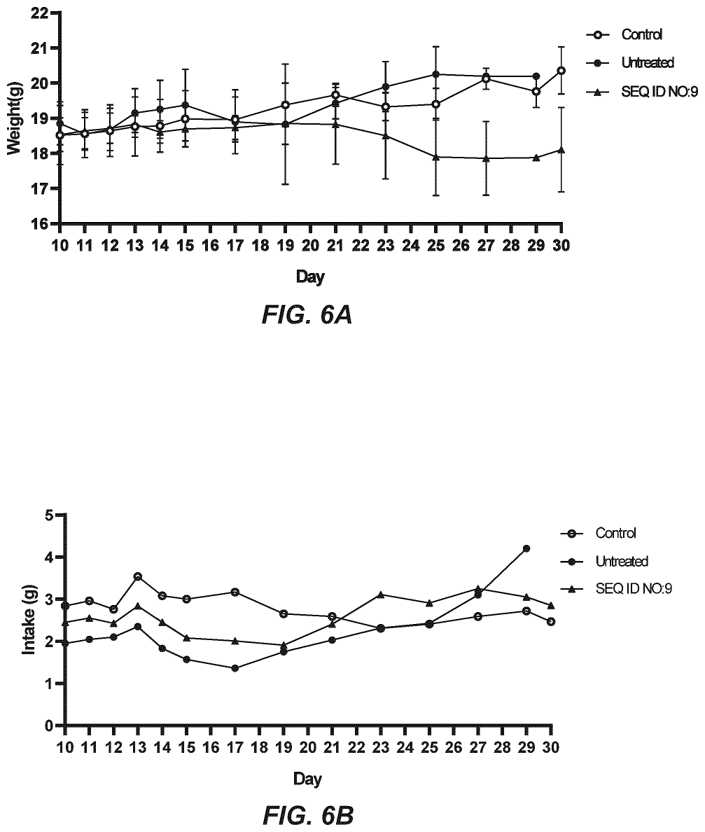

The experiment demonstrated that SEQ ID NO:9 significantly inhibited unilateral (right side) tumor growth in vivo compared to the saline control group (panel) (FIGS. 5A-5D). In addition, there was no significant change in the body weight and food intake of the mice during the treatment of SEQ ID NO:9, which means that SEQ ID NO:9 may not have toxic effects in vivo. The reason for the weight loss after treatment was presumed to be caused by a change in the excessive swelling of the tumor (FIGS. 6A-6B).

### Example 7

### Inhibiting Murine Breast Cancer Growth in Allograft Mouse Model and Prolongs the Survival In Vivo

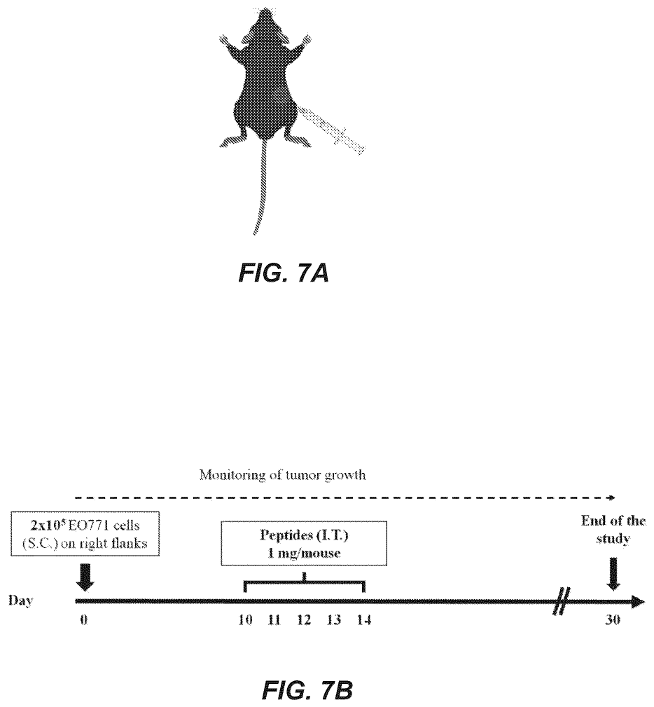

To evaluate the effect of SEQ ID NO:9 in vivo, the E0771 allogeneic model was used in C57BL/6 mice (FIGS. 7A-7B). E0771 tumor cells were harvested, and washed in medium and injected subcutaneously (S.C.) into the right flank of C57BL/6 mice (2×10⁵E0771 cells per mouse/50 μl RPMI-1640).

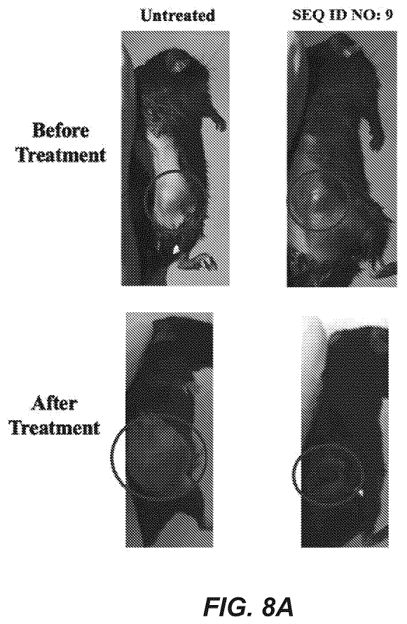

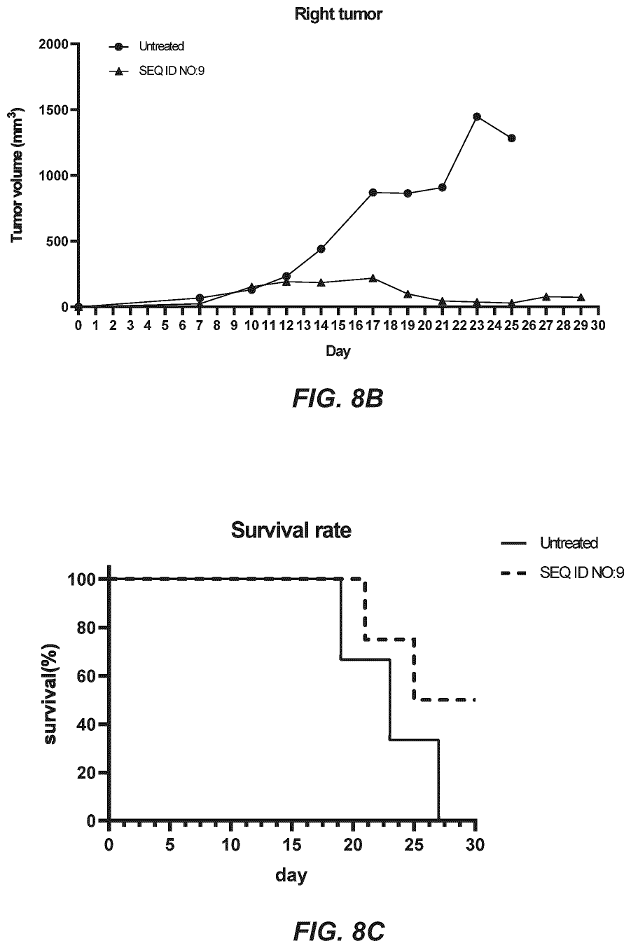

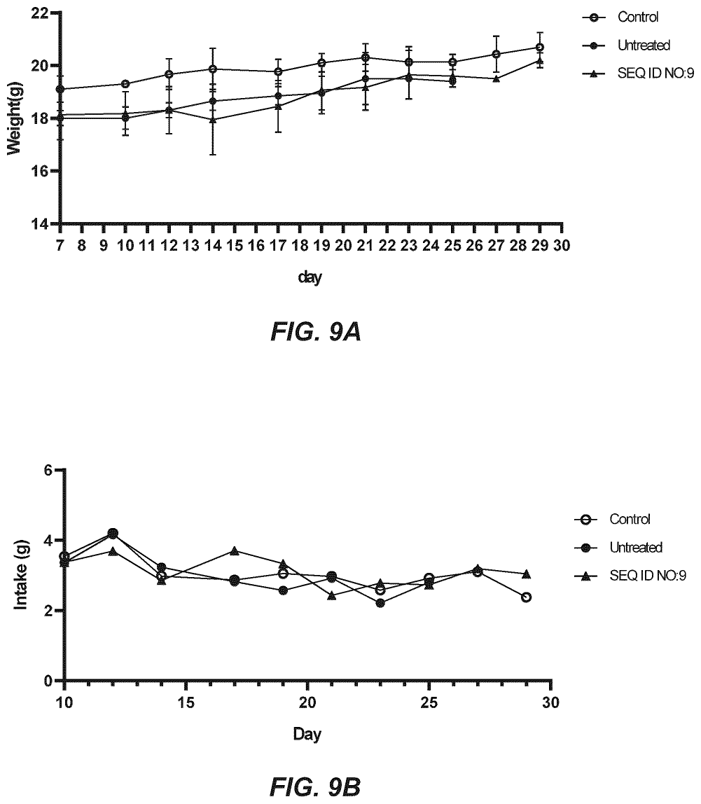

The experiment demonstrated that SEQ ID NO:9 significantly inhibited tumor growth in vivo compared to the saline control group (FIGS. 8A-8C). In addition, there was no significant change in the body weight and food intake of the mice during the treatment of SEQ ID NO:9, which means that SEQ ID NO:9 may not have toxic effects in vivo (FIGS. 9A-9B).

To sum up, SEQ ID NO:9 in the disclosure extended the lifespan of tumor-bearing mice (FIG. 2D, FIG. 5D and FIG. 9C). These results indicated that SEQ ID NO:9 could significantly inhibit tumor growth and improve the survival rate of tumor-bearing C57BL/6 mice.

The foregoing is illustrative of the present invention, and is not to be construed as limiting thereof. The invention is defined by the following claims, with equivalents of the claims to be included therein.

1. An oncolytic peptide of formula (I),
Xₘ-(AC₁X)ₙ-(DLC₂)X_{q}
wherein X is selected from basic amino acids;
wherein A is selected from aromatic amino acids;
wherein C₁ or C₂ is selected from the group consisting of basic amino acids and non-polar amino acids;
wherein D is selected from the group consisting of aromatic amino acids and non-natural amino acids; and
wherein L is leucine.

2. The oncolytic peptide according to claim 1, wherein the basic amino acids are selected from the group consisting of lysine, arginine, and histidine.

3. The oncolytic peptide according to claim 2, wherein m is 4 to 8.

4. The oncolytic peptide according to claim 2, wherein q is 0 to 2.

5. The oncolytic peptide according to claim 2, wherein n is 0 to 2.

6. The oncolytic peptide according to claim 5, wherein the aromatic amino acids are selected from the group consisting of tryptophan, phenylalanine, and tyrosine.

7. The oncolytic peptide according to claim 5, wherein the non-polar amino acids are selected from the group consisting of leucine, alanine, valine, isoleucine, proline, phenylalanine, methionine, and tryptophan.

8. The oncolytic peptide according to claim 5, wherein the non-natural amino acids are selected from the group consisting of β-naphthylalanine (Nal), (benzothien-3-yl) alanine (Bal), diphenylalanine (Dip), (4,4′-biphen-yl) alanine (Bip), (anthracen-9-yl) alanine (Ath) and (2,5,7-tri-tert-butyl-indol-3-yl) alanine (Tht).

9. The oncolytic peptide according to claim 8, wherein a C-terminus of the oncolytic peptide is formed in part by a modification selected from the group consisting of amidation, acetylation, formylation, hydroxylation, lipid modification, methylation and phosphorylation.

10. The oncolytic peptide according to claim 9, wherein the oncolytic peptide is selected from the group consisting of SEQ ID NO: 1, SEQ ID NO:2, SEQ ID NO: 3, SEQ ID NO:4, SEQ ID NO: 5, SEQ ID NO: 6, SEQ ID NO:7, SEQ ID NO:8, SEQ ID NO:9 and SEQ ID NO: 10.

11. A method comprising: administering a pharmaceutical composition comprising the oncolytic peptide of claim 1 in a therapeutically effective amount to a subject in need thereof.

12. The method according to claim 11, wherein the subject is diagnosed with a cancer.

13. The method of claim 12, wherein the cancer comprises at least one of melanoma, fibrosarcoma, prostate cancer, breast cancer, uterine cancer, leukemia, ovarian cancer, endometrial cancer, cervical cancer, colorectal cancer, testicular cancer, lymphoma, rhabdomyosarcoma, neuroblastoma, pancreatic cancer, lung cancer, brain tumor, skin cancer, stomach cancer, oral cancer, liver cancer, laryngeal cancer, gallbladder cancer, thyroid cancer, liver cancer, kidney cancer, or nasopharyngeai carcinoma.

14. The method of claim 11, wherein the subject is a mammal.

15. The method of claim 14, wherein the mammal is selected from the group consisting of a cat, dog, rabbit, cattle, horse, sheep, goat, monkey, mouse, rat, gerbil, guinea pig, pig and a human.

16. The oncolytic peptide according to claim 1, wherein the oncolytic peptide is administered in a therapeutically effective amount to a subject in need thereof.

17. The oncolytic peptide according to claim 16, wherein the subject is diagnosed with a cancer.

18. The oncolytic peptide according to claim 17, wherein the cancer comprises at least one of melanoma, fibrosarcoma, prostate cancer, breast cancer, uterine cancer, leukemia, ovarian cancer, endometrial cancer, cervical cancer, colorectal cancer, testicular cancer, lymphoma, rhabdomyosarcoma, neuroblastoma, pancreatic cancer, lung cancer, brain tumor, skin cancer, stomach cancer, oral cancer, liver cancer, laryngeal cancer, gallbladder cancer, thyroid cancer, liver cancer, kidney cancer, or nasopharyngeal carcinoma.

19. The oncolytic peptide according to claim 18, wherein the subject is a mammal.

20. The oncolytic peptide according to claim 19, wherein the mammal is selected from the group consisting of a cat, dog, rabbit, cattle, horse, sheep, goat, monkey, mouse, rat, gerbil, guinea pig, pig and a human.
